(19) United States
(12) Patent Application Publication
(10) Pub. No.: US 2026/0191576 A1
(43) Pub. Date: Jul. 9, 2026

(54) DEVICE FOR MANIPULATING AN INTRAMEDULLARY ROD, COMPRISING A THREADED ELEMENT FOR CONNECTING AN INTRAMEDULLARY ROD

(71) Applicant: JABEZ MEDICAL GMBH & CO. KG, Glattbach (DE)

(72) Inventor: Siegbert Gregel, Edertal (DE)

(21) Appl. No.: 19/099,444

(22) PCT Filed: Jul. 28, 2023
(86) PCT No.: PCT/DE2023/100561

§ 371 Date: Mar. 3, 2026

(30) Foreign Application Priority Data
Jul. 30, 2022 (DE) 202022104343.4

(51) Int. Cl. A61B 17/88; A61B 17/72; A61B 90/00

(52) U.S. Cl. CPC A61B 17/8872; A61B 17/72; A61B 90/08; A61B 2090/0811

(57) ABSTRACT

In an embodiment a device extending in an axial longitudinal direction and having a proximal end and a distal end includes a shaft assigned to an actuating element, wherein the distal end of the shaft coincides with the distal end of the device, and wherein the shaft comprises, at the distal end, an engagement section configured for engaging and rotating or retaining a complementary engagement section of an intramedullary nail, which is arrangeable oppositely to the device in the longitudinal direction, and a threaded element arranged at the distal end of the shaft, which is rotatable relative to the shaft about the longitudinal direction, and which is screwable onto a complementary thread of the intramedullary nail.

### CROSS-REFERENCE TO RELATED APPLICATIONS

This patent application is a national phase filing under section 371 of PCT/DE2023/100561, filed Jul. 28, 2023, which claims the priority of German utility model application 20 2022 104 343.4, filed Jul. 30, 2022, each of which is incorporated herein by reference in its entirety.

### TECHNICAL FIELD

The invention relates firstly to a device for actuating an intramedullary nail. The invention further relates to a set comprising the device and a threaded element.

### BACKGROUND

Techniques and devices for intramedullary nailing in case of bone fractures of the extremities were first significantly developed and described by Küntscher et al., see Küntscher, G., Maatz, R., “Technik der Marknagelung”, Georg Thieme Verlag Leipzig, 1945.

Simpler intramedullary nails for interlocking nailing are known, but their installation during surgery can only be checked by means of intraoperative X-ray imaging. This is, due to the duration, stressful not only for the patient but in particular for the surgical staff. Furthermore, such simple intramedullary nails must be secured in the bone using transverse screws, which requires complex targeting devices, in particular for long femurs, but also for humeri and tibiae, in order to be able to place such transverse screws precisely through the skin with as little damage to the skin as possible. These additional surgical wounds that have to be inflicted into an extremity have to be treated after surgery, but also after surgical removal of the transverse screws, which causes additional stress for the patient.

WO 2006/066536 A1 discloses an intramedullary nail, in particular for the closed reposition of a fracture of a tubular bone by insertion into a drill hole in its medullary cavity, which is of comparatively simple construction and can be used easily, quickly and safely with only minimal use of intraoperative X-rays, without requiring interventions in unhurt areas of an extremity. The intramedullary nail comprises a nail core, at the front region of which a head piece is provided, at which the origin of a strip is fixed. The strip is wound around the nail core and is fixed at its end in the rear region around the nail core in a rotatably lockable manner, and the strip expands the radial contour of the intramedullary nail when it is rotated in the opposite direction to the direction in which it is wound.

Advantageous further developments of this intramedullary nail with a nail core are the subject of publications WO 2014/060576 A1 and WO 2014/060578 A1.

WO 2015/117996 A1 discloses a further intramedullary nail, namely an assembly for the internal stabilization and/or fixing of an elongate hollow body, in particular a bone, such as a tubular bone, or a prosthesis, with a stabilizing element with an elongate first element comprising a helical shape, the proximal end of which is connected to a first fastening element, and from the distal end of which a second element extends, which is connected to a second fastening element in the region of the first fastening element, wherein the first element and the second element preferably merge into one another connectionless as sections of the stabilizing element, wherein an outer fastening element forming the first fastening element clampingly fixes an inner fastening element forming the second fastening element, so that the first fastening element cannot be twisted relative to the second fastening element.

Other comparable intramedullary nails are shown in DE 10 2018 005 690 A1 and DE 20 2020 003 051 U1.

A tool, which is here referred to as a device for actuating an intramedullary nail or a tool, is required for tensioning a spring element of such an intramedullary nail, namely for reducing its cross-section in order to be able to insert it into the medullary cavity, and for detensioning the spring element in order to be able to clamp the spring element in the inserted state with respect to the inner surface of the medullary cavity, and not least also for handling the intramedullary nail both during implantation and during explantation.

DE 20 2007 008 234 U1 discloses a mounting instrument for an intramedullary nail comprising at least one spiral spring body (spring element) which can be expanded within the intramedullary nail channel by a relative rotatory movement. On the mounting instrument there is provided a handle (actuating element) for a positioning element and at least one rotary handle (actuating element) which is connected to a spiral spring body rotary element tube (shaft) which can be rotated relative to the positioning element. This publication discloses a device of the kind mentioned at the beginning. This known device for actuating an intramedullary nail comprises a total of three shafts which are rotatable inside each other, with each shaft being assigned to an actuating element. An inner shaft serves to hold the nail core of an intramedullary nail against unwanted rotation, a middle shaft serves to actuate and/or to hold an inner spring element, and an outer shaft serves to actuate and/or hold an outer spring element of the intramedullary nail. A disadvantage of this device lies in the fact that the actuation of a total of three actuating elements during surgery is complicated, and several people may be needed in order to perform an actuation simultaneously.

WO 2015/117996 A1, already mentioned in relation to an intramedullary nail, also shows a device for opposing rotation of two, in particular helical, elements (spring elements) of an assembly for internal stabilization, wherein the device comprises a holding section with a first hollow cylinder section, preferably with a first handle (actuating element), wherein the first hollow cylinder section coaxially surrounds a second hollow cylinder section (shaft) and a shaft-shaped or hollow cylinder-shaped inner section (shaft). This publication also shows a device of the kind mentioned at the beginning.

A common disadvantage of the known devices for actuating an intramedullary nail lies in the fact that only little and not precisely definable axial forces and torque can be transmitted between the tool placed on the intramedullary nail and the intramedullary nail itself, and there is always a risk of the tool slipping off the intramedullary nail. This is particularly disadvantageous during the implantation or explantation of the intramedullary nail, which may even have to be performed via a kink through a lateral opening of the medullary canal.

### SUMMARY

Embodiments provide a device for actuating an intramedullary nail with which axial forces and/or a torsional moment can be transmitted between the device and an intramedullary nail in a defined manner.

Embodiments provide a generic device that is designed and further developed by a threaded element arranged at the distal end of the shaft, which is rotatable relative to the shaft about the axial longitudinal direction, and which is configured to be screwed onto a complementary thread of an intramedullary nail which is arrangeable oppositely to the device in the longitudinal direction.

Embodiments provide a device for actuating an intramedullary nail, wherein the device extends in an axial longitudinal direction and comprises a proximal end and a distal end, and comprises a shaft which is assigned to an actuating element, wherein the distal end of the shaft essentially coincides with the distal end of the device, and wherein the shaft comprises at its distal end an engagement section for engaging and rotating or retaining a complementary engagement section of an intramedullary nail which is arrangeable oppositely to the device in the longitudinal direction.

A set according to the invention comprises a device mentioned at the beginning and a threaded element according to the invention.

The present invention provides a device for actuating an intramedullary nail, which comprises a threaded element that, in order to connect an intramedullary nail to the device, can be screwed onto a suitable thread of the intramedullary nail that is arrangeable or is arranged oppositely to the device in the longitudinal direction. This allows the device to be connected to an intramedullary nail quickly and reversibly, but at the same time firmly with respect to the axial direction. The intramedullary nail connected in this way can thus be handled in an ideal way during implantation and also during explantation. Furthermore, the invention allows the intramedullary nail to be connected to the device in a rotatably fixed manner. An additional shaft of a prior device for actuating an intramedullary nail, which shaft is intended to prevent undesirable rotating of a component of the intramedullary nail, can thus be dispensed with.

After completion of the actuation, the threaded element proposed by the invention can be easily unscrewed from the complimentary thread of the intramedullary nail and can then remain on the device. The intramedullary nail implanted during the actuation can then remain at the implantation site, or the intramedullary nail explanted during the actuation can be discarded, respectively.

The device according to the invention for actuating an intramedullary nail is suitable, inter alia, for the intramedullary nails disclosed in the aforementioned patent documents, provided that they comprise a suitable thread on their side usually facing the tool. Preferably, this is an external thread, and more preferably a thread in a so-called intramedullary nail head.

In particular, an intramedullary nail which is suitable for the present invention is described in the aforementioned WO 2015/117996 A1, where the suitable thread of the intramedullary nail is designated by reference number 44. FIGS. 4, 5, 8, and 9 of that publication, together with the description associated therewith, are hereby incorporated by reference into the present application.

An intramedullary nail suitable to be actuated by the present device according to the invention is further described in the German utility model application 20 2021 106 260.6, meanwhile published as DE 20 2021 106 260 U1, of the present applicant. The content of that application is hereby incorporated by reference in its entirety into the present application.

In the context of the present invention, the terms “proximal” and “distal” are always understood from the perspective of the person, resp. the surgeon, who actuates and handles an intramedullary nail.

Here, a device for actuating an intramedullary nail is also referred to as a “tool” without deviating from the inventive concept.

In the following, the aspects according to the invention are discussed further, for which purpose reference is made in part to non-limiting advantageous embodiments and further developments of the invention. The features of advantageous further developments can be realized individually or in any combination, whereby further advantageous embodiments of the invention are created.

Throughout the present discussion of configurations, further developments, examples and embodiments of the devices according to the invention, methods of using the devices and uses of the devices according to the invention are also described. These methods and uses also expressly lie within the scope of the present invention.

In a first preferred embodiment of the device according to the invention, the threaded element is designed as a sleeve-like element which surrounds the shaft at its distal end.

Preferably, the threaded element is displaceable relative to the shaft and within the longitudinal direction.

The threaded element preferably is displaceable by screwing or sliding the threaded element.

If no screwing of the threaded element relative to the shaft is provided, a positive locking between the threaded element and the shaft may preferably be provided, so that at least an axial longitudinal force can be induced from a connected intramedullary nail via the threaded element into the shaft of the device, or vice versa.

In a specific further preferred embodiment of the device, the distal end of the shaft comprises an external thread, and the threaded element is provided with a corresponding internal thread, and the threaded element is displaceable relative to the distal end of the shaft along the longitudinal direction by a screwing motion of the threaded element.

The threaded element preferably is displaceable beyond the distal end of the shaft by a screwing motion relative to the shaft. This means in particular that at least one distal end of the threaded element can be displaced beyond the distal end of the shaft. Thus, the threaded element can be screwed onto an intramedullary nail, which is arranged oppositely to the device in the longitudinal direction, in a particularly practical manner.

In a particularly preferred embodiment, the threaded element is a threaded sleeve. It is further preferred that the sleeve comprises at least one internal thread, and it is also preferred that the threaded sleeve surrounds the area of the distal end of the shaft at least partially, seen in the axial direction. As viewed in the circumferential direction, the threaded sleeve is preferably closed, but may also comprise recesses, if this appears appropriate.

A threaded sleeve provided as a threaded element may feature a division of the thread into two partial threads.

The two partial threads may merge into one another, or the two partial threads may comprise an axial distance between each other.

Preferably, the two partial threads of a threaded sleeve which is configured as a threaded element have opposite directions of rotation. This makes it possible to automatically bring the distal end of the device and the proximal end of a connecting or connected intramedullary nail closer together by screwing the threaded sleeve, and, if necessary, makes it even possible to brace them against each other in the axial direction.

As an alternative to a threaded sleeve, the threaded element may be a union nut.

A threaded element with a central passage and an internal thread is preferably employed as a union nut, with one side of the union nut comprising a reduced diameter of its central passage. Such a reduction of the internal diameter may also be preferably designed as a shoulder, or referred to as such.

A union nut provided as a threaded element of the device is preferably arranged such that its shoulder faces away from the distal end of the shaft. In other words, the union nut can transmit a force to the shaft via the shoulder on its proximal side, while the connection to the intramedullary nail is established on the distal side of the union nut.

Preferably, the shaft of the device comprises a widening (collar) in the vicinity of its distal end. Such a collar can preferably serve as the point of force transmission between the shoulder of a union nut and the shaft of the device.

In another preferred embodiment of the device, a union nut is arranged on the shaft in such a way that a pulling, directed to the distal end of the shaft, of the union nut from the shaft is prevented by a positive fit between a collar of the shaft and a shoulder of the union nut. At the same time, a longitudinal axial displaceability of the union nut in the direction of the proximal end of the device may still be possible.

In a further preferred development, a union nut is configured such that, as seen in the longitudinal direction, it embraces a collar of the shaft from both sides, so that there is no significant or no axial displaceability of the union nut. This allows a defined axial position of the threaded element to be achieved, which does not change even when an intramedullary nail is being connected.

The shaft of the device according to the invention may be an actuating shaft. Such an actuating shaft may extend as a continuous element between the actuating element associated to the shaft, and the distal end of the shaft.

In this context, it is therefore preferable that the distal end of the shaft coincides with the distal end of the actuating shaft.

In an alternative embodiment, the shaft comprises an actuating shaft and an adapter piece connected thereto in the longitudinal direction. In other words, the shaft may comprise, between the associated actuating element and its distal end, an actuating shaft as well as an adapter piece connected thereto. Such an adapter piece may in particular be interchangeable and serve to actuate intramedullary nails of different diameters with a single tool, namely by using the appropriate adapter piece for each intramedullary nail size.

Preferably, the distal end of the shaft coincides with the distal end of the adapter piece.

In other words, the present invention can be implemented both in a device with a continuous actuating shaft being the shaft, but also in a device with a shaft consisting of an actuating shaft and an adapter piece, without deviating from the general idea of the invention.

A further specific and preferred development of the device according to the invention is characterized in that the distal end of the shaft comprises, in its engagement section, teeth, which taper as seen in the longitudinal direction facing away from the shaft, so that, when engagement is established with a sleeve of an intramedullary nail which is arrangeable oppositely to the device in the longitudinal direction, with the sleeve comprising, in an engagement section, recesses, which taper in a corresponding manner as seen in the longitudinal direction facing away from the shaft, and, where applicable, merge into slots oriented in the longitudinal direction, the teeth can cause an expansion of the sleeve of the intramedullary nail, wherein an external thread is formed at the distal end of the shaft, and wherein the threaded element is a threaded sleeve, and comprises at a proximal end a first internal thread which engages with the external thread of the shaft, and comprises at a distal end a second internal thread which runs in the opposite direction to the first internal thread, and which is configured for connecting an intramedullary nail.

This preferred further development of the device according to the invention is in particular suitable for the intramedullary nail already mentioned above and also described in WO 2015/117996 A1.

In a further preferred embodiment of the device, the teeth of the engagement section taper at a pointed angle as seen in the longitudinal direction facing away from the shaft.

Preferably, there is a longitudinal axial distance between the first internal thread and the second internal thread of the threaded element.

In a particularly preferred further embodiment of this aspect of the invention, the shaft comprises a first marking for setting a first longitudinal axial position of the threaded element, in which a longitudinal axial and rotatably fixed engagement between the engagement section of the shaft and the engagement section of the intramedullary nail is present. This allows for a safe handling of the connected intramedullary nail without, on the other hand, an undesirable rotatability of the two sleeves (anchor elements) together with the respectively associated spring element.

For an explanation of the structure and function of such an intramedullary nail, which is not the subject of this document, reference is made to WO 2015/117996 A1.

In this first setting of the threaded sleeve relative to the shaft of the device, which is predetermined by the first marking, the intramedullary nail connected to the device can then be implanted or explanted in a longitudinally axial and rotatably fixed manner, without the tensioning of the two spring elements, which is directed against each other, having been released. In particular, implantation can thus be carried out with an outer and inner spring element of the intramedullary nail each being tensioned in the winding direction, so that the outer diameter of the intramedullary nail is reduced.

Further preferred, and alternatively or additionally, the shaft comprises a second marking for setting a second longitudinal axial position of the threaded element, in which the engagement section of the shaft and the engagement section of the intramedullary nail are tensioned against each other in the longitudinal axial direction in such a way that the sleeve of the intramedullary nail is expanded. This second marking can be set in particular when the intramedullary nail has been inserted in the correct position into the medullary canal. The inner sleeve of the intramedullary nail with the inner spring element anchored therein can then rotate relative to the outer sleeve of the intramedullary nail with the outer spring element anchored therein, and the spring elements each can detension and come to lay against the inner wall of the medullary canal. In addition, the inner spring element can then be expanded by means of the device via the inner sleeve, i.e. by rotating the inner sleeve relative to the outer sleeve in the opposite winding direction, so that both spring elements can be tensioned against the inner wall. This position can then be secured by rotating the threaded element back to the first marking, as the outer sleeve, which will be no longer expanded, will then clamp the inner sleeve again.

In this aspect of the invention also, the threaded element, preferably a threaded sleeve, may comprise a section with a reduced diameter of its central passage (shoulder), which prevents the threaded element from being pulled off the shaft in the direction of the distal end of the shaft.

Preferably, the shaft comprises a widening (collar) in the vicinity of its distal end, which prevents the threaded sleeve from being pulled off the shaft in the direction of the distal end of the shaft.

It is particularly preferred that a collar of the shaft is provided as a stop for a shoulder of the threaded sleeve.

### BRIEF DESCRIPTION OF THE DRAWINGS

Supplementary or in addition to the advantageous embodiments and further developments of the teaching already discussed, embodiments of devices, uses and methods according to the invention shown in the drawings to the extent of FIGS. 1 to 10 are explained in more detail below. However, the examples discussed with reference to the drawings do not limit the invention to the examples shown. In discussing the embodiments with reference to the drawing, generally preferred embodiments and further developments of the teaching are also shown.

Further developments of the advantageous embodiments described above with features of the embodiments described below, just like further developments of the embodiments described below with features of the embodiments described above, expressly form further advantageous embodiments of the invention, and thus form part of the present disclosure.

With regard to the following explanation of the figures, it should generally be noted that, for reasons of clarity, reference signs already shown in previous figures and already explained above in this respect have in some cases not been repeated in subsequent figures and/or are in some cases not explained again on the basis of these subsequent figures. For the explanation of such reference signs and the associated technical features, reference is therefore made in full to the respective description of the corresponding preceding figures in order to avoid repetition.

FIG. 1 is a perspective view of a first intramedullary nail for the actuation of which the device according to the invention is adapted;

FIG. 2 is a lateral sectional view of a first preferred embodiment of the device according to the invention for actuating an intramedullary nail;

FIG. 3 is the corresponding side view of the device of FIG. 2;

FIG. 4 is a perspective view of the device of FIG. 2 and FIG. 3 as seen from the distal end;

FIG. 5a is a detailed perspective view illustrating the approach and coupling of the distal end of the device according to the invention from FIGS. 2 and 3 to the proximal end of a suitable intramedullary nail according to FIG. 1;

FIG. 5b is a view as in FIG. 5a, but viewed after coupling of the parts;

FIG. 6 is a perspective view of a second preferred embodiment of the device according to the invention for actuating an intramedullary nail;

FIG. 7 is a perspective view of further preferred embodiments of the device according to the invention for actuating an intramedullary nail, wherein two alternative embodiments, namely a third and a fourth embodiment, are combined in one representation;

FIG. 8a is an enlarged sectional view of detail X from FIG. 7, which illustrates a third preferred embodiment of the device according to the invention;

FIG. 8b is an enlarged sectional view of an alternative design of detail X from FIG. 7, which illustrates a fourth preferred embodiment of the device according to the invention;

FIG. 9 is a perspective view of the proximal end of a second intramedullary nail known from the prior art, for the actuation of which the device according to the invention is adapted; and

FIG. 10 is a perspective view of the proximal end of the intramedullary nail from FIG. 9 in an engagement with the distal end of a fifth preferred embodiment of the device according to the invention.

### DETAILED DESCRIPTION OF ILLUSTRATIVE EMBODIMENTS

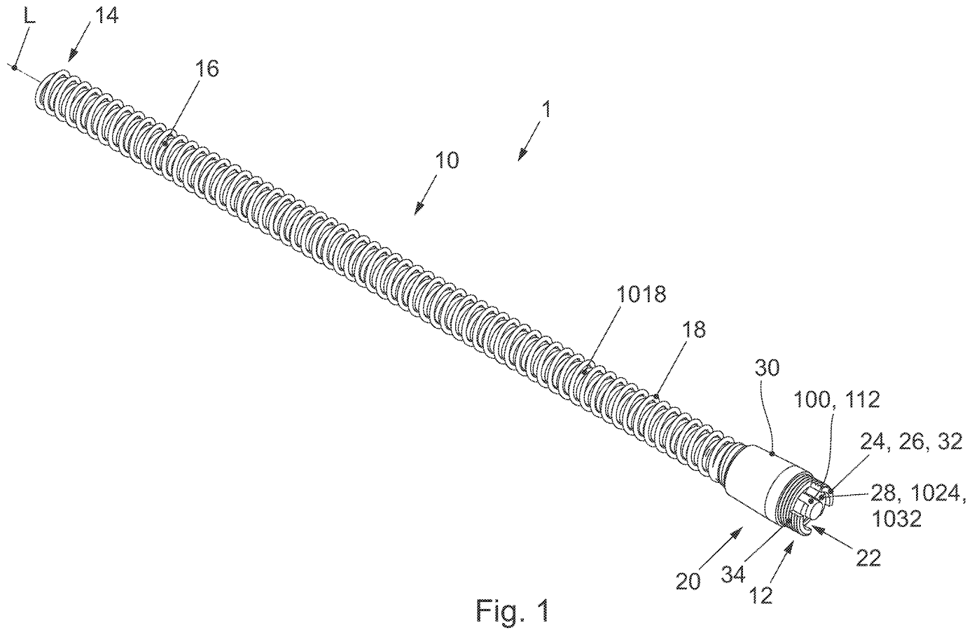

FIG. 1 shows a perspective view of an intramedullary nail 1, for the actuation of which the device according to the invention for actuating an intramedullary nail 1 is adapted. The illustration of this intramedullary nail 1 serves to provide a further understanding and illustration of the teaching according to the invention here.

The intramedullary nail 1 shown is the subject of the German utility model application 20 2021 106 260.6, meanwhile published as DE 20 2021 106 260 U1, by the present applicant. The figures and figure description therein are hereby incorporated by reference into the present figure description.

The intramedullary nail 1 comprises a sheath element 10 and a nail core 100. The nail core 100 is of no further relevance in the present case, namely for the actuation of the intramedullary nail 1. In view of this fact, the sheath element 10 is also referred to below as intramedullary nail 1.

The intramedullary nail 1 is intended for the internal stabilization and/or fixing of an elongate hollow body, in particular a bone. It extends in a longitudinal direction L and comprises a proximal end 12 and a distal end 14.

The intramedullary nail 1 comprises a substantially cylindrical interior 16, wherein an outer contour of the intramedullary nail 1, viewed perpendicularly to the longitudinal direction L, comprises an elongate spring element 18 which, starting from the proximal end 12 and with respect to the longitudinal direction L, is wound in a helical shape.

The radial extension of the wound spring element 18 can be reduced by tensioning the elongate spring element 18 in the winding direction, and can be increased by detensioning the elongate spring element 18 counter to the winding direction.

Here, the spring element 18 essentially conceals the view on the interior 16, since the outer contour of the intramedullary nail 1 is essentially formed by the wound spring element 18. The spring element 18 is wound in the form of a three-dimensional coil, and here in particular in the form of a cylindrical coil.

The diameter of the intramedullary nail 1 can be reduced by tensioning the spring element 18. This is done in particular in order to be able to insert the intramedullary nail 1 into the medullary canal. On the other hand, the intramedullary nail 1 can also be braced, by expanding its outer contour, namely by detensioning the spring element 18, relative to the inner wall of an elongate hollow body to be stabilized, in particular in the medullary canal of a tubular bone.

In this embodiment, the intramedullary nail 1 comprises not just one spring element 18, but two elongate spring elements 18, 1018, arranged one above the other, which are each wound in a helical shape with respect to the longitudinal direction L of the intramedullary nail 1. For this purpose, a second spring element 1018 extends within the spring element 18 or first spring element 18.

Here, the two superimposed spring elements 18, 1018 form a three-dimensional double spiral, namely a double helix. The two spring elements 18, 1018 arranged one above the other are here wound in opposite directions compared to each other, and consist of one continuous element which merges, at the distal end 14, from the first spring element 18 into the second spring element 1018.

The proximal end 12 of the intramedullary nail 1 comprises a nail head 20. Both the proximal end of the first spring element 18 and the proximal end of the second spring element 1018 are anchored to the nail head 20. Furthermore, the nail head 20 comprises an axial aperture 22 for inserting the nail core 100.

The proximal end of the first spring element 18 is anchored in a first anchor element 24. The first anchor element 24 can also be referred to here as the outer anchor element 24, which is an outer sleeve 26.

The proximal end of the second spring element 1018 is anchored in a second anchor element 1024. The second anchor element 1024 can also be referred to here as the inner anchor element 1024, which is an inner sleeve 28.

The locking sleeve 30, which is arranged on the very outside, secures the anchoring of the outer, first spring element 18 to the first anchor element 24, i.e. to the outer sleeve 26. The locking sleeve 30 also conceals the further view of this section.

The inner sleeve 28 can be rotated relative to the outer sleeve 26. By twisting the inner sleeve 28 in the winding direction of the second spring element 1018, the spring element 1018 is tensioned and thus, with the outer sleeve 26 held in place or even twisted in the opposite direction, also tensions the first spring element 18 via the distal transition into the first spring element 18. The diameter of the intramedullary nail 1 is reduced.

By twisting the inner sleeve 28 against the winding direction of the second spring element 1018, the second spring element 1018 is detensioned and thus also detensions the first spring element 18 via the distal transition into the first spring element 18. For this purpose, it may already be sufficient to remove the previously applied torque in order to tension the second spring element 1018 from the inner sleeve 28. The second spring element 1018 may then already detension itself automatically, thereby rotating the inner sleeve 28 against the winding direction.

As a result, the outer spring element 18 can contact the inside of the medullary canal. Finally, the inner spring element 1018 may also contact the inside of the first spring element 18 in order to stabilize and secure it.

Alternatively or additionally, by twisting the outer sleeve 26, and thus the nail head 20, in the winding direction of the first spring element 18, the outer spring element 18 can be tensioned and thus the diameter of the intramedullary nail 1 may be reduced, and vice versa. The inner sleeve 28 should then be retained in place or even be actuated into the opposite direction.

In this illustration, the axial aperture 22 in the proximal end 12 of the intramedullary nail 1, namely in the nail head 20, can also be seen. Viewed from the inside out, the nail core 100 is surrounded by the inner sleeve 28, the outer sleeve 26, and the locking sleeve 30.

The design of the outer sleeve 26, which is recessed as viewed in the circumferential direction, provides a first engagement section 32 in the first anchor element 24. The design of the inner sleeve 28, which is recessed as viewed in the circumferential direction, provides a second engagement section 1032 in the second anchor element 1024. Both engagement sections 32, 1032 are formed in the form of axially extending teeth and provide useful engagement sections 32, 1032 for complementary engagement sections of an oppositely arrangeable device according to the invention for actuating an intramedullary nail 1.

Thus, for each spring element 18, 1018 in the intramedullary nail 1, a respective possibility for engagement is provided for a device according to the invention for actuating an intramedullary nail 1, namely for tensioning or detensioning the spring elements 18, 1018 of the intramedullary nail 1.

In addition to the engagement section 32, an external thread 34 is also provided in the outer sleeve 26. This thread 34 is suitable for connecting the intramedullary nail 1 to a device according to the invention, which will be explained below.

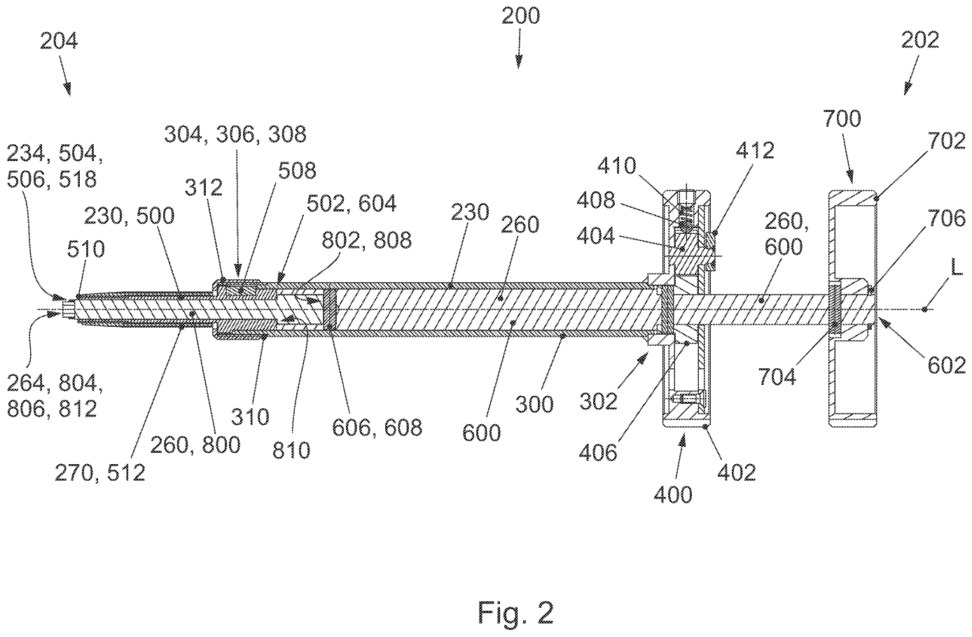

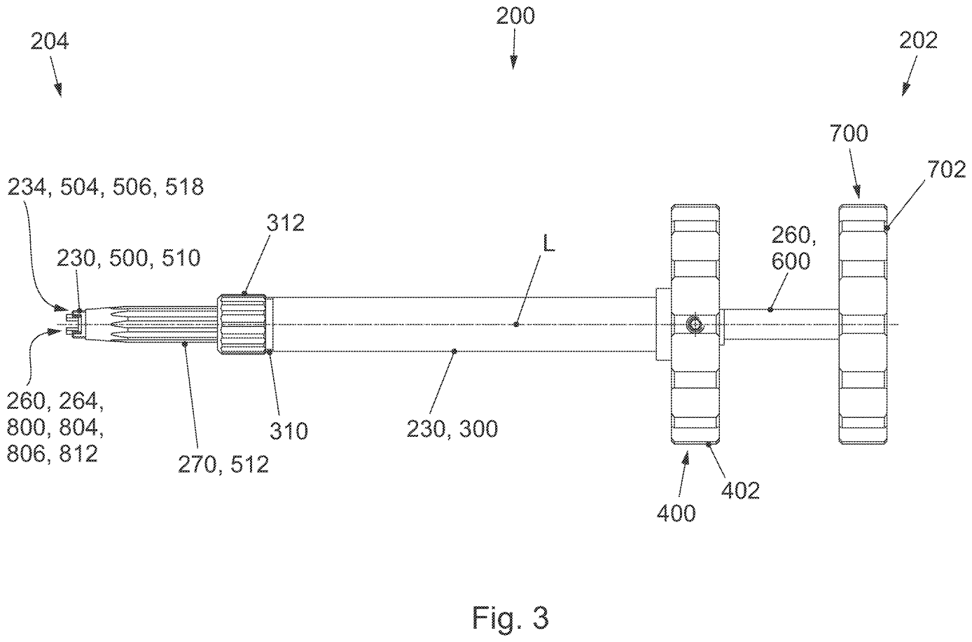

FIG. 2 shows a lateral sectional view of a first preferred embodiment of the device 200 according to the invention for actuating an intramedullary nail 1, and FIG. 3 shows the corresponding lateral view of this device 200.

The device 200 for actuating an intramedullary nail 1 extends in the axial longitudinal direction L and comprises a proximal end 202 and a distal end 204.

A first, outer shaft 230 is associated with a first, distal actuating element 400. The distal end 234 of the outer shaft 230 essentially coincides with the distal end 204 of the device 200. The shaft 230 thus extends between the first actuating element 400 and the distal end 204 of the device 200.

This first, outer shaft 230 comprises at its distal end 234 an engagement section 506 for engaging and rotating or retaining a complementary engagement section 32 of an intramedullary nail 1 which is arrangeable oppositely to the device 200 in the longitudinal direction L.

Here, the first, outer shaft 230 comprises a first, outer actuating shaft 300 and a first, outer adapter piece 500. The adapter piece 500 is interchangeable and serves to adapt the device 200 to intramedullary nails 1 with different diameters.

The first outer actuating shaft 300 is assigned to the first distal actuating element 400. The outer actuating shaft 300 extends in the longitudinal direction L between a proximal end 302 and a distal end 304, and comprises at its distal end 304 an engagement section 306 for engaging and rotating, or retaining, a complementary counterpart.

The first outer adapter piece 500 is arranged between the distal end 304 of the first, outer actuating shaft 300 and the distal end 204 of the device 200, and the adapter piece 500 is, at the distal end 304 of the outer actuating shaft 300, non-rotatably connected to the engagement section 306 of the actuating shaft 300.

The adapter piece 500 comprises a proximal end 502 and a distal end 504, and extends therebetween in the longitudinal direction L, namely between the distal end 304 of the actuating shaft 300 and the distal end 234 of the outer shaft 230, or the distal end 204 of the device 200. The outer adapter piece 500 comprises, at the distal end 204 of the device 200, an engagement section 506, which is complementary to an engagement section 32 of the anchor element 24 of an intramedullary nail 1 that is arrangeable oppositely.

Here, the device 200 comprises two shafts 230, 260 for indirectly actuating two spring elements 18, 1018 of an intramedullary nail 1 that can be arranged oppositely. The two shafts 230, 260 are arranged concentrically.

A second, inner shaft 260 is associated with a second, proximal actuating element 700. Just as well as the first shaft 230, the distal end 264 of the inner shaft 260 also essentially coincides with the distal end 204 of the device 200. The inner shaft 260 thus extends between the second actuating element 700 and the distal end 204 of the device 200.

This second, inner shaft 260 comprises at its distal end 264 an engagement section 806 for engaging and rotating or retaining a complementary engagement section 1032 of an intramedullary nail 1 which is arrangeable oppositely to the device 200 in the longitudinal direction L.

Here, the second, inner shaft 260 comprises a second, inner actuating shaft 600 and a second, inner adapter piece 800. The inner adapter piece 800 is also interchangeable and, like the outer adapter piece 500 already described, serves to adapt the device 200 to intramedullary nails 1 of different diameters.

The second, inner actuating shaft 600 is associated with the second, proximal actuating element 700. The inner actuating shaft 600 extends in the longitudinal direction L between a proximal end 602 and a distal end 604, and comprises at its distal end 604 an engagement section 606 for engaging in and rotating or retaining a complementary counterpart.

The second, inner adapter piece 800 is arranged between the distal end 604 of the second, inner actuating shaft 600 and the distal end 204 of the device 200, which, at the distal end 604 of the inner actuating shaft 600, is connected to the engagement section 606 of the actuating shaft 600 in a rotatably fixed manner.

The second, inner adapter piece 800 has a proximal end 802 and a distal end 804 and extends in the longitudinal direction L between the distal end 604 of the actuating shaft 600 and the distal end 264 of the inner shaft 260, or the distal end 204 of the device 200. The inner adapter piece 800 comprises, at the distal end 204 of the device 200, an engagement section 806, which is complementary to an engagement section 1032 of the anchor element 1024 of an intramedullary nail 1 that can be arranged oppositely.

Thus, an adapter piece 500, 800 is arranged here between the distal end 304, 604 of each actuating shaft 300, 600 and the distal end 204 of the device 200. Thus, two adapter pieces 500, 800 are provided here, wherein the adapter pieces 500, 800 are arranged concentrically.

The first, outer actuating shaft 300 is formed as a sleeve, and the second, inner actuating shaft 600 is formed as a shaft rotatably supported therein. Further, the first, outer adapter piece 500 is formed as a sleeve, and the second, inner adapter piece 800 is formed as a shaft rotatably supported therein.

The distal ends 504, 804 of the adapter pieces 500, 800 with their respective engagement sections 506, 806 thereby substantially coincide with the distal end 204 of the device 200.

Each engagement section 506, 806 of the adapter pieces 500, 800 thereby comprises axially extending recesses or protrusions, which may also be referred to as teeth 518, 812.

The respective engagement between the actuating shafts 300, 600 and the respectively associated adapter piece 500, 800 is explained below.

The distal end 604 of the second, inner actuating shaft 600 is provided with a radially extending pin 608. On the other hand, the opposite proximal end 802 of the second, inner adapter piece 800, comprises a radially extending groove 808, which can engage the pin 608 in a rotatably fixed manner. During assembly, the second adapter piece 800 can thus be pushed onto the distal end 604 of the second, inner actuating shaft 600, or vice versa, and the groove 808 engages around the pin 608, whereby a non-rotatable but axially releasable coupling is established between the inner actuating shaft 600 and the inner adapter piece 800.

The distal end 304 of the first, outer actuating shaft 300, on the other hand, comprises an axially extending groove 308 as an engagement section 306, and the proximal end 502 of the first, outer adapter piece 500 comprises an axially extending key 508, which engages in the groove 308 in a rotatably fixed manner.

The assembly comprising the inner actuating shaft 600 and the inner adapter piece 800 remains rotatable relative to the assembly comprising the outer actuating shaft 300 and the outer adapter piece 500. For assembly, the inner adapter piece 800 is inserted with its distal end 804 through the proximal end 502 of the outer adapter piece. Then, this assembly is inserted into the distal end 304 of the outer actuator shaft 300, so that the couplings of the key 508 and groove 308 and the groove 808 and pin 608 are established.

The distal end 304 of the first, outer actuating shaft 300 further comprises an external thread 310, and after insertion into the distal end 304, the two adapter pieces 500, 800 can be fixed in or on the actuating shaft 300 by the lock nut 312 screwed onto the external thread 310.

According to the invention, a threaded element 270 is arranged here at the distal end 234 of the outer shaft 230, which is rotatable relative to the outer shaft 230 about the axial longitudinal direction L, and which is configured to be screwed onto a complementary thread 34 of an intramedullary nail 1 which is arrangeable oppositely to the device 200 in the longitudinal direction L.

For this purpose, here the outer adapter piece 500 comprises at its distal end 504, and thus the outer shaft 230 comprises at its distal end 234, an external thread 510 in addition to the engagement section 506.

The threaded element 270, configured here as a threaded sleeve 512, is screwed onto the external thread 510. In a particularly advantageous manner, the threaded sleeve 512 can be screwed onto a matching external thread 34, here in an appropriate manner an external thread 34 in the nail head 20 or in the outer sleeve 26 of an intramedullary nail 1 when coupling an intramedullary nail 1 to the device 200. In this way, not only can the spring element 18, 1018 of the intramedullary nail 1 be actuated with the device 200, but the intramedullary nail 1 can also be handled in a particularly advantageous manner with the device 200 during implantation or explantation, since it can be securely and firmly fixed to the device 200 with respect to the axial direction L.

In addition to an axial fixation of the intramedullary nail 1 by screwing the threaded element 270 onto the thread 34, the intramedullary nail can be connected in a rotatably fixed manner (torsion-resistant). This can be achieved preferably, but not exclusively, by screwing the threaded element 270 onto a suitable stop at the distal end of the thread 34 of the intramedullary nail 1.

The first, distal actuating element 400 is formed as a handwheel 402, and the second, proximal actuating element 700 is formed as a handwheel 702 spaced therefrom. The center of each handwheel 402, 702 coincides with the longitudinal axis L.

The first, distal actuating element 400 is firmly connected to the first, outer actuating shaft 300. The first actuating element 400 is used to hold the first actuating shaft 300 in place or to rotate it if necessary.

The second, proximal actuating element 700 is connected to the second, inner actuating shaft 600 via the pin 704 and the retaining ring 706. The second actuating element 700 is used to rotate the second actuating shaft 600 relative to the first actuating shaft 300 or, if necessary, to hold it in place.

For said rotatability of the second actuating shaft 600 relative to the first actuating shaft 300, a ratchet mechanism is implemented in the distal handwheel 402, whereby one direction of rotation can be locked in each case in the sense of a ratchet mechanism. Here, a ratchet 404 acts on a pinion 406. In the desired direction of rotation, the ratchet slides on the teeth of the pinion 406, but in the opposite direction, the ratchet 404 jams in the teeth and locks the rotation. Such a mechanism is known in itself.

The ratchet 404 is subjected to the force of a compression spring 410 via a ball 408. The respective direction of rotation can be set via a ratchet lever 412 by pivoting the ratchet 404 about the longitudinal axis L. Depending on the inclined positioning of the ratchet 404, it will lock the respective backward rotation of the inner actuating shaft 600.

It should be emphasized at this point that FIGS. 1 to 5 provide a description of a first preferred embodiment of the invention, without however limiting the invention thereto. Thus, without deviating from the inventive concept, it is possible, if deemed appropriate, to also assemble a threaded element 270 according to the invention at the distal end 264 of the inner shaft 260.

Furthermore, the invention is not limited to the presence of adapter pieces 500, 800. If no adaptation option for the device 200 is to be implemented, the adapters 500, 800 can also be omitted, in which case the shafts 230, 260 can then be formed solely by the actuating shafts 300, 600. The threaded element 270 according to the invention can then be assembled at the distal end 304 of the outer actuating shaft 300, or at the distal end 604 of the inner actuating shaft 600.

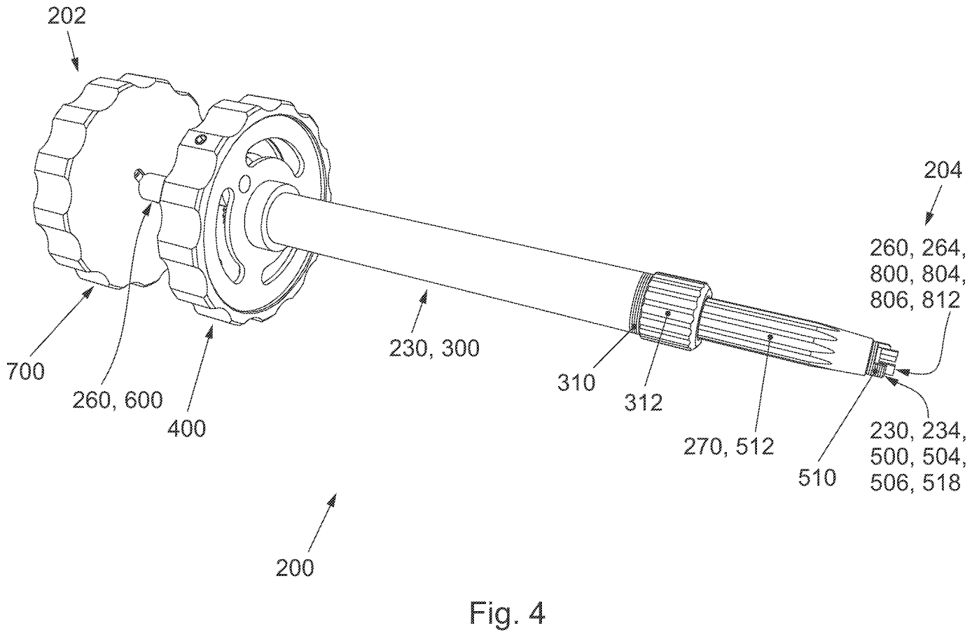

FIG. 4 shows a perspective view of the device 200 from FIG. 2 and FIG. 3, as seen from the distal end 204. The following items can be seen from the outside (starting from the proximal end 202 and advancing to the distal end 204): proximal actuating element 700, inner shaft 260 with inner actuating shaft 600, distal actuating element 400, outer shaft 230 with outer actuating shaft 300, external thread 310 of the outer actuating shaft 300, lock nut 312, threaded element 270, here as threaded sleeve 512, distal end 234 of the outer shaft 230 with the distal end 504 of the outer adapter piece 500, comprising the engagement section 506 on the outside with the teeth 518, and distal end 264 of the inner shaft 260 with the distal end 804 of the inner adapter piece 800, which comprises the engagement section 806 on the inside with the teeth 812.

It is clearly visible here that the threaded element 270 can be screwed without any problems beyond the distal end 204 of the device in the direction of an intramedullary nail 1 which is arrangeable oppositely.

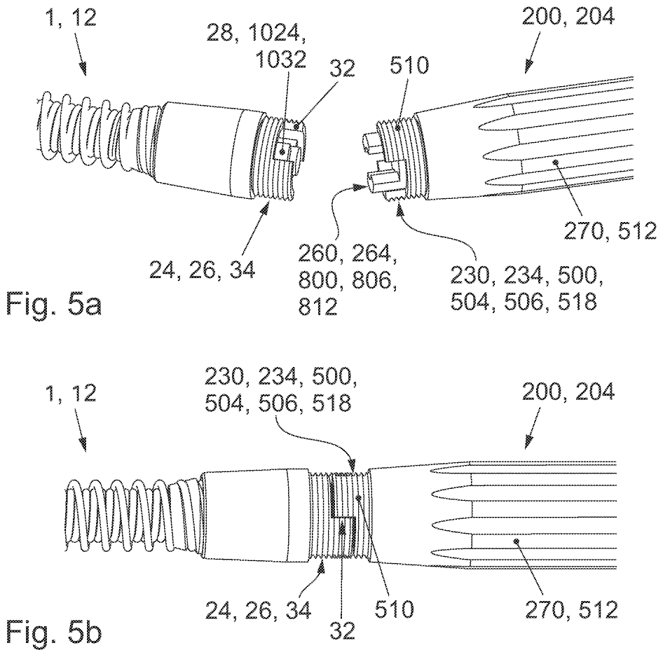

FIG. 5a shows a detailed perspective view illustrating the approach and coupling of the distal end 204 of the device 200 according to the invention shown in FIGS. 2, 3, and 4, to the proximal end 12 of a suitable intramedullary nail 1 according to FIG. 1, and FIG. 5b shows a corresponding view after the coupling took place.

The outer engagement section 506 of the outer adapter piece 500 is brought into contact with the outer engagement section 32 of the intramedullary nail 1, and the inner engagement section 806 of the inner adapter piece 800 is brought into contact with the inner engagement section 1032 of the intramedullary nail 1.

The inner teeth 812 engage with the inner engagement section 1032. The outer teeth 518 are formed as segment-like recesses or teeth 518 in the external thread 510, and the outer engagement section 32 of the intramedullary nail 1 is formed to be exactly complementary to this. This allows the teeth 518 to interlock with the outer sleeve 26 of the intramedullary nail 1, and at the same time a continuous external thread 510 is formed, onto which the threaded element 270, in this case the threaded sleeve 512, can then be screwed. The intramedullary nail 1 is fixed axially immediately by screwing it on and can then be operated and handled by the device 200. At the latest when the threaded sleeve 512 hits the stop at the distal end of the thread 34 of the intramedullary nail 1, the intramedullary nail 1 is then also fixed in a rotatably fixed manner to the device 200.

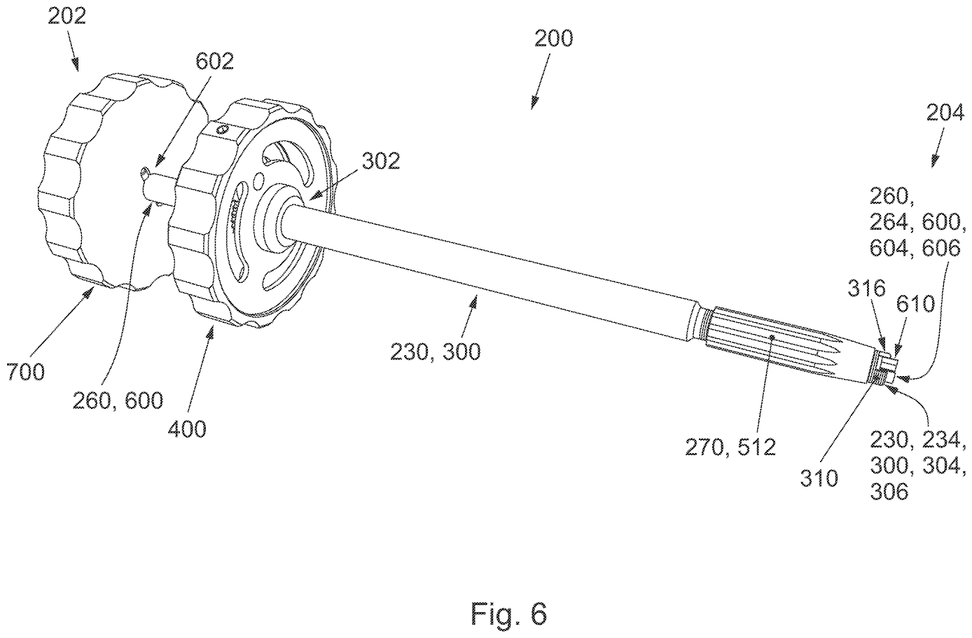

FIG. 6 shows a perspective view of a second preferred embodiment of the device 200 according to the invention for actuating an intramedullary nail. In contrast to the embodiment shown in FIGS. 1 to 5, no adapters 500, 800 are provided here for adapting the device 200 to intramedullary nails 1 with different diameters. Apart from that, the mode of operation of the embodiment shown in FIG. 6 corresponds to that of the example shown in FIGS. 1 to 5.

The device 200 for actuating an intramedullary nail 1 extends in the axial longitudinal direction L and comprises a proximal end 202 and a distal end 204.

A first, outer shaft 230 is assigned to a first, distal actuating element 400. The distal end 234 of the outer shaft 230 essentially coincides with the distal end 204 of the device 200.

The shaft 230 thus extends between the first actuating element 400 and the distal end 204 of the device 200.

Here, the first outer shaft 230 consists solely of the first outer actuating shaft 300, as no adapters 500, 800 are provided here. Thus, the outer actuating shaft 300 also extends between the first actuating element 400 (proximal end 302) and the distal end 204 of the device 200, and the distal end 304 of the outer actuating shaft 300 coincides with both the distal end 234 of the outer shaft 230 and the distal end 204 of the device 200.

The second, inner shaft 260 is assigned to a second, proximal actuating element 700. In a manner corresponding to the first shaft 230, the second inner shaft 260 consists solely of the second inner actuating shaft 600, since no adapters 500, 800 are provided here. Thus, the inner actuating shaft 600 extends between the second actuating element 700 (proximal end 602) and the distal end 204 of the device 200, and the distal end 604 of the inner actuating shaft 600 coincides with both the distal end 264 of the inner shaft 260 and the distal end 204 of the device 200.

The first, outer actuating shaft 300 (corresponding to the outer shaft 230) comprises at its distal end 304 an engagement section 306 for engaging and rotating or retaining a complementary engagement section 32 of an intramedullary nail 1 which is arrangeable oppositely to the device 200 in the longitudinal direction L.

The second, inner actuating shaft 600 (corresponding to the inner shaft 260) comprises at its distal end 604 an engagement section 606 for engaging and rotating or retaining a complementary engagement section 1032 of the intramedullary nail 1.

Each engagement section 306, 606 of the actuating shafts 300, 600 comprises axially extending recesses or projections, which can in the present case be referred to as teeth 316 of the engagement section 306, or as teeth 610 of the engagement section 600, respectively.

According to the invention, a threaded element 270 is arranged at the distal end 234 of the outer shaft 230, namely at the distal end 304 of the outer actuating shaft 300, which is rotatable relative to the outer shaft 230 about the axial longitudinal direction L, and which is configured to be screwed onto a complementary thread 34 of a intramedullary nail 1 which is arrangeable oppositely to the device 200 in the longitudinal direction L.

For this purpose, the outer actuating shaft 300 comprises at its distal end 304 an external thread 310, and thus the outer shaft 230 comprises at its distal end 234 in addition to the engagement section 306 also an external thread 310.

The threaded element 270, here as a threaded sleeve 512, is screwed onto the external thread 310. The threaded sleeve 512 can be screwed onto a matching thread 34, here as an appropriate external thread 34, and preferably in the nail head 20 or in the outer sleeve 26 of an intramedullary nail 1, in a particularly advantageous manner when coupling an intramedullary nail 1 to the device 200. This allows an axial and/or a rotatably fixed connection to be established between an intramedullary nail 1 and the device 200.

For further details, reference can be made to the descriptions of the first embodiment in FIGS. 1 to 5. The same applies to the mode of operation of the actuating elements 400, 700 and the actuating shafts 300, 600 actuated thereby.

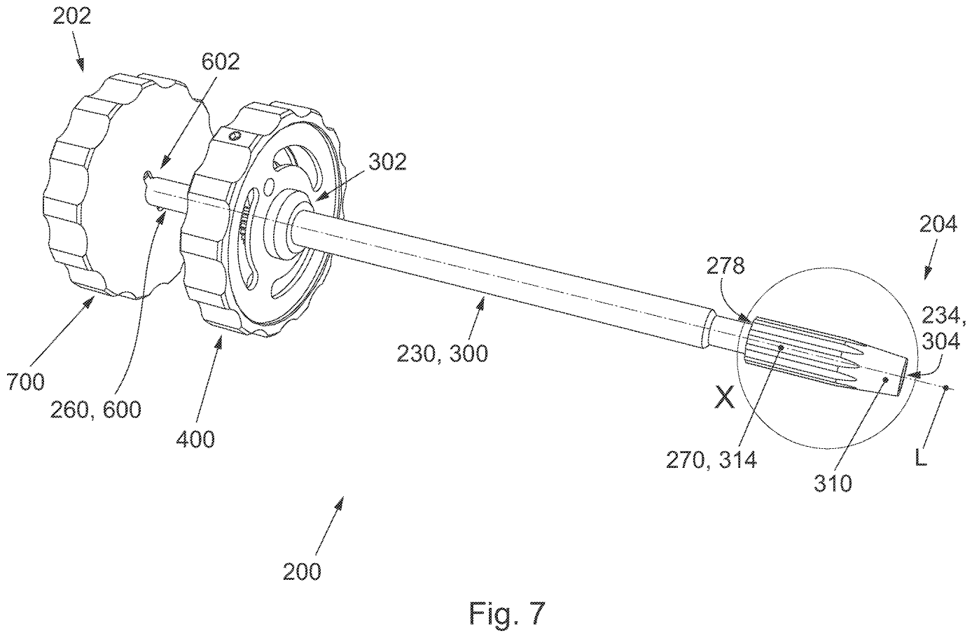

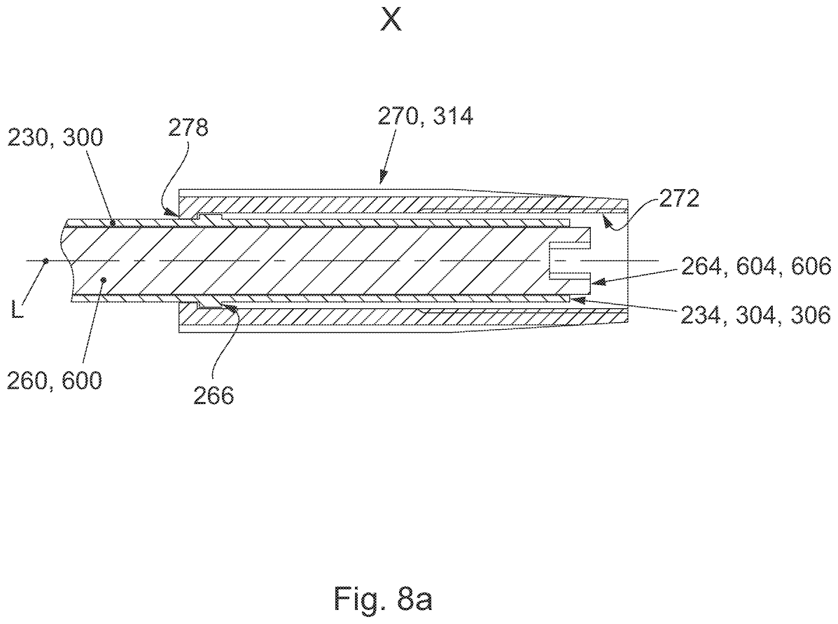

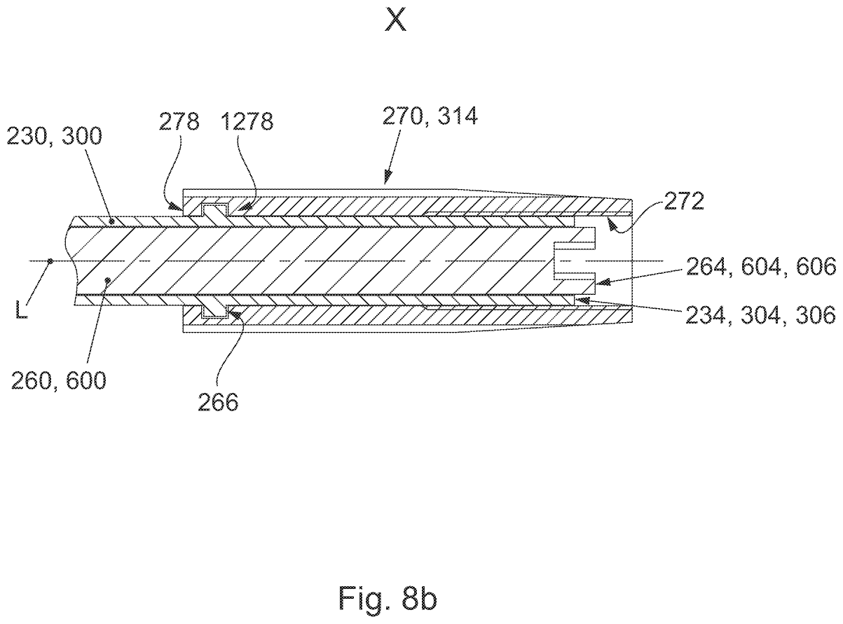

FIG. 7 shows a perspective view of further preferred embodiments of the device 200 according to the invention for actuating an intramedullary nail 1, wherein two alternative embodiments, namely a third embodiment (see FIG. 8a) and a fourth embodiment (see FIG. 8b), are combined in one illustration.

Except for the distal end 204, the device 200 as shown in FIG. 7 corresponds to the embodiment shown and described in FIG. 6. Here, too, no adapters 500, 800 are provided. In particular, with regard to the structure and interaction of the actuating elements 400, 700 and the actuating shafts 300, 600, reference can be made to the explanations already given with respect to FIG. 6.

A threaded element 270, which is designed as a union nut 314, is arranged at the distal end 234 of the outer shaft 230, namely at the distal end 304 of the outer actuating shaft 300. The union nut 314 features, on its side facing away from the distal end 204 of the device 200, which is also the side facing away from the distal end 234 of the outer shaft 230, a reduced diameter of its central passage. This lateral constriction is referred to here as a shoulder 278.

The union nut 314 is secured to the distal end 234 of the shaft by means of a positive locking, so that an external thread 310 (see FIG. 6) in the outer actuating shaft 300 can be omitted here.

Since the positive locking only acts in the longitudinal axial direction L, the union nut 314 can be rotated relative to the outer shaft 230 (outer actuating shaft 300) about the axial longitudinal direction L. This allows the internal thread 272 of the union nut 314 to be screwed onto a complementary thread 34 of an intramedullary nail 1 which can be arranged oppositely to the device 200 in the longitudinal direction L.

The engagement sections 306, 606 of the actuating shafts 300, 600 can be designed as shown in FIG. 6.

FIG. 8a shows an enlarged sectional view of detail X from FIG. 7, which illustrates a third preferred embodiment of the device 200 according to the invention.

The outer shaft 230, namely the outer actuating shaft 300, comprises a widening, namely a collar 266, in the vicinity of its distal end 234 or 304. The shoulder 278 of the union nut 314 encompasses the collar 266 on the proximal side. This allows the union nut 314 to be freely rotated about the longitudinal axis L, and also to be displaced in the longitudinal direction L, namely here to be shifted, in the direction of the proximal end 202 of the device 200.

However, removal of the threaded element 270, namely the union nut 314, from the shaft 230 in the direction of the distal end 234 of the outer shaft 230 is prevented by the positive locking between the collar 266 of the shaft 230 and the shoulder 278 of the threaded sleeve 314.

FIG. 8b shows an enlarged sectional view of an alternative design of detail X from FIG. 7, which illustrates a fourth preferred embodiment of the device 200 according to the invention.

Here, when viewed in the longitudinal direction L, the union nut 314 comprises two adjacent shoulders 278, 1278. Thus, as viewed in the longitudinal direction L, the union nut 314 embraces the collar 266 of the outer shaft 230 from both sides, so that the threaded element 270, namely the union nut 314, can no longer be displaced in the longitudinal axis. At the same time, the union nut 314 can be rotated and screwed about the longitudinal direction L. An intramedullary nail 1 which is oppositely arranged can be pulled toward the device 200 by screwing on the union nut 314, so that a longitudinal axial displaceability of the union nut 314 is not necessary.

From a manufacturing or assembling point of view, one of the shoulders 278, 1278 could be provided by a circlip. Such a circlip could be inserted into the union nut 314 when it is located in its intended position on the collar 266 of the shaft 230.

In this embodiment, the threaded element 270 has a fixed position along the device 200, and thus a screwing-on operation onto an oppositely arranged intramedullary nail 1 can always take place under the same conditions.

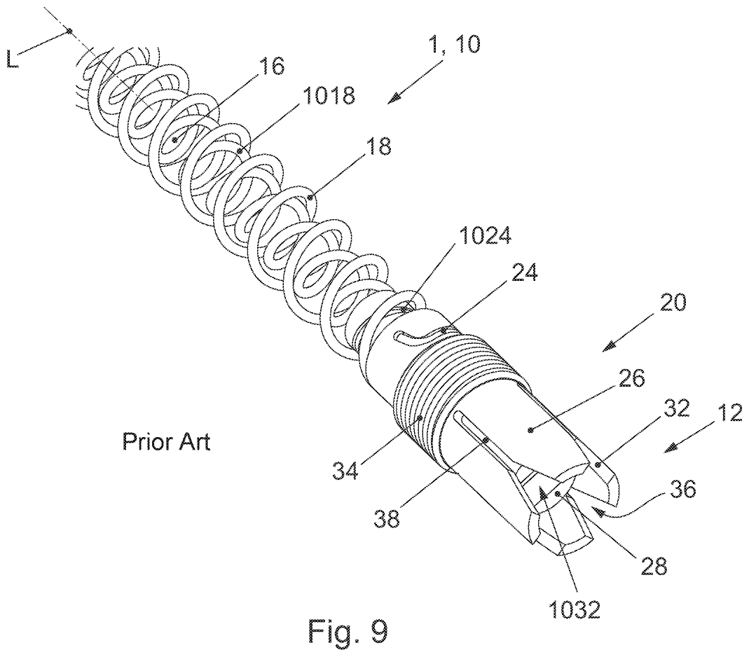

FIG. 9 shows a perspective view of the proximal end 12 of a second intramedullary nail 1 known from the prior art, for which the device 200 according to the invention is suitable. This intramedullary nail 1 is known from WO 2015/117996 A1, in particular from FIG. 4 therein.

Like the example in FIG. 1, the intramedullary nail 1 comprises a sheath element 10 with an interior 16 and a nail head 20. A nail core 100 is not provided here.

Furthermore, this example also comprises two elongated spring elements 18, 1018 arranged one above the other, which are wound in a helical shape relative to the longitudinal direction L of the intramedullary nail 1. A second spring element 1018 runs inside the spring element 18 or first spring element 18.

The two spring elements 18, 1018 arranged one above the other are, relative to each other, wound in opposite directions, and consist of one continuous element, which transitions at the distal end from the first spring element 18 to the second spring element 1018.

The proximal end of the first spring element 18 is anchored in a first anchor element 24. The first anchor element 24 can here also be referred to as the outer anchor element 24, as it consists of an outer sleeve 26.

The proximal end of the second spring element 1018 is anchored in a second anchor element 1024. The second anchor element 1024 can here also be referred to as the inner anchor element 1024, as it consists of an inner sleeve 28.

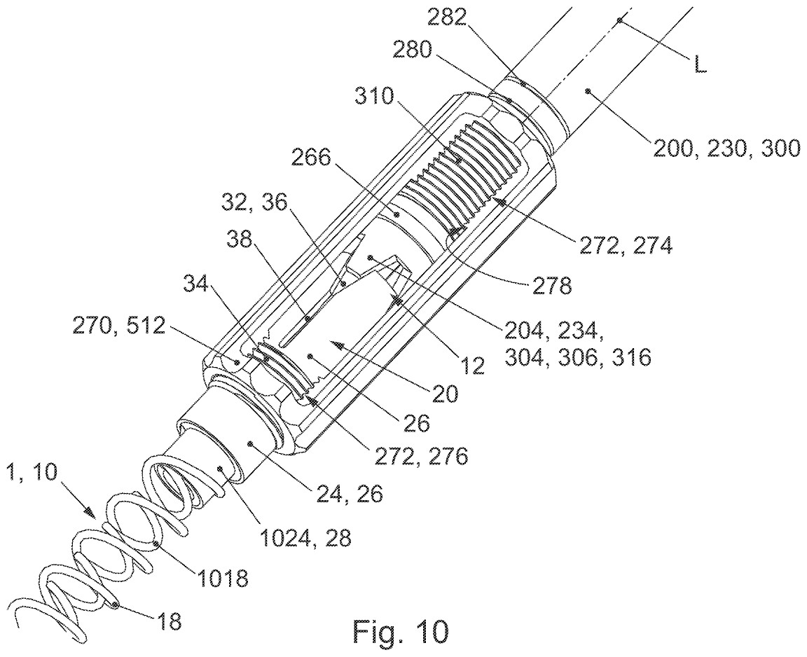

A locking sleeve 30 (see FIG. 1) arranged at the very outside is not shown here. In addition to an engagement section 32, an external thread 34 is also provided in the outer sleeve 26. This thread 34 is suitable for connecting the intramedullary nail 1 to a device 200 according to the invention (see FIG. 10).

As in the example shown in FIG. 1, the inner sleeve 28 can be rotated relative to the outer sleeve 26. By rotating the inner sleeve 28 in the winding direction of the second spring element 1018, the second spring element 1018 and thus also the first spring element 18 are tensioned, and the diameter of the intramedullary nail 1 is reduced, and vice versa. Reference is made to the explanations in FIG. 1.

The non-continuous design of the outer sleeve 26, as viewed in the circumferential direction, provides a first engagement section 32 in the first anchor element 24. The proximal end of the inner sleeve 28, configured here as a radially extending bar, provides a second engagement section 1032 in the second anchor element 1024.

In this example of an intramedullary nail 1, it is specific that the outer sleeve 26 is preloaded relative to the inner sleeve 28 in the radial direction. Therefore, the outer sleeve 26 clamps the position of the inner sleeve 26 in place unless it is actuated. A current state of tension between the spring elements 18 and 1028 is therefore maintained when an actuation of the outer sleeve 26 is terminated.

For this purpose, the first engagement section 32 in the outer sleeve 26 is formed as axially extending recesses 36 between adjacent teeth. These recesses 36 taper at a pointed angle as seen in the longitudinal direction L facing away from the proximal end 12, and then merge into slots 38 which are oriented in the longitudinal direction L.

In the case of a longitudinal axial engagement between a device 200 with a correspondingly shaped engagement section 306 and the recesses 36 of the intramedullary nail 1, and by the application of a longitudinal axial force, the outer sleeve 26 can thus be expanded, thereby releasing the clamping between the outer sleeve 26 and the inner sleeve 28. The inner sleeve 28 can then rotate relative to the outer sleeve 26, or can be rotated.

FIG. 10 shows a perspective view of the proximal end 12 of the intramedullary nail 1 from FIG. 9 in engagement with the distal end 204 of a fifth preferred embodiment of the device 200 according to the invention.

The distal end 204 of the device 200 is shown in engagement with the proximal end 12 of the intramedullary nail 1. A threaded sleeve 512, which is screwed onto both ends 204, 12, serves as the threaded element 270 here. The threaded sleeve 512 comprises an internal thread 272 that is comprised of a first partial thread 274 and a second partial thread 276, which have opposite directions of rotation, and which are axially spaced apart from each other. The first partial thread 274 is assigned to the external thread 310 of the outer shaft 230 of the device 200, and the second partial thread 276 is assigned to the external thread 34 of the intramedullary nail 1.

The outer shaft 230 of the device 200 again corresponds to the outer actuating shaft 300. The distal end 304 of the outer actuating shaft 300 coincides with the distal end 234 of the outer shaft 230, and also with the distal end 204 of the device 200.

The distal end 234 of the outer shaft 230 comprises in its engagement section 306 teeth 316, which taper as seen facing away from the shaft 230 in the longitudinal direction L. By establishing an engagement between the teeth 316 of the device 200 and the recesses 36 of the intramedullary nail 1, at first a rotatably fixed connection is provided, due to the complementary design of the engagement sections 306, 32. The screwed-on threaded sleeve 512 further provides a firm axial connection between the intramedullary nail 1 and the device 200.

The recesses 36 of the intramedullary nail 1 taper as seen in the longitudinal direction L facing away from the shaft 230, matching the shape of the teeth 316, and further transition into slots 38 in the outer sleeve 26, which slots 38 are oriented in the longitudinal direction L.

By further screwing the threaded sleeve 512, the partial threads 274, 276, the intramedullary nail 1, and the device 200 are further pulled together, so that the distal end 204 of the device 200 with the teeth 316 exerts a longitudinal axial force on the proximal end 12 of the intramedullary nail with the tapered recesses 36.

This consequently causes the outer sleeve 26 of the intramedullary nail 1 to expand, as the teeth 316 will push the recesses 36 and the slots 38 apart.

With the device 200 shown, two specifically distinguishable operating states of the intramedullary nail 1 can be achieved. Each of these operating states is assigned a marking 280, 282 on the outer shaft 230 of the device 200.

A first marking 280 serves to set a first longitudinal axial position of the threaded element 270 and indicates the operating state in which a longitudinal axial and rotatably fixed engagement between the engagement section 306 of the shaft 230 and the engagement section 32 of the intramedullary nail 1 is present, but wherein the clamping between the outer sleeve 26 and the inner sleeve 28 has not yet been released. In this operating state, the intramedullary nail 1 can, for example, in a very advantageous manner be implanted in a medullary canal, even through narrow or off-center openings to the medullary canal, by being in a state of tensioning (i.e. reduction in diameter) of the spring elements 18, 1018.

A second marking 282 serves to set a second longitudinal axial position of the threaded element 270 and indicates the operating state in which the engagement section 306 of the shaft 230 and the engagement section 32 of the intramedullary nail 1 are tensioned against each other in the longitudinal axial direction L in such a way that the outer sleeve 26 of the intramedullary nail 1 is expanded by the teeth 316 of the device 200. The clamping between the outer sleeve 26 and the inner sleeve 28 is then released, and the spring elements 18, 1018 of the intramedullary nail 1 can detension and, for example, come to lay against the inner wall of the medullary canal, or can be actuated as desired by using the device 200.

### Embodiments

Supplementary or in addition to the advantageous embodiments, further developments and examples already discussed, the invention is described below with reference to further specific preferred embodiments, which, however, do not limit the invention to the embodiments described. These embodiments are expressly part of the present description.

Further developments of the advantageous embodiments and examples described above with features of the embodiments described below, just like further developments of the embodiments described below with features of the embodiments and examples described above, expressly form further advantageous embodiments of the invention, and thus form part of the present disclosure.

Embodiment 1: Device for actuating an intramedullary nail, wherein the device extends in an axial longitudinal direction and comprises a proximal end and a distal end, and comprises a shaft which is assigned to an actuating element, wherein the distal end of the shaft essentially coincides with the distal end of the device, and wherein the shaft comprises at its distal end an engagement section for engaging and rotating or retaining a complementary engagement section of an intramedullary nail which is arrangeable oppositely to the device in the longitudinal direction.

Embodiment 2: Device according to the preceding embodiment, comprising a threaded element arranged at the distal end of the shaft, which is rotatable relative to the shaft about the axial longitudinal direction, and which is configured to be screwed onto a complementary thread of an intramedullary nail which is arrangeable oppositely to the device in the longitudinal direction.

Embodiment 3: Device according to the preceding embodiment, wherein the threaded element is designed as a sleeve-like element which surrounds the shaft at its distal end.

Embodiment 4: Device according to one or both of the two preceding embodiments, wherein the threaded element is displaceable relative to the shaft in the longitudinal direction.

Embodiment 5: Device according to the preceding embodiment, wherein the threaded element is displaceable by screwing or sliding the threaded element.

Embodiment 6: Device according to one or more of the preceding embodiments, wherein the distal end of the shaft comprises an external thread, and the threaded element is provided with a corresponding internal thread, and is displaceable relative to the distal end of the shaft along the longitudinal direction by a screwing motion.

Embodiment 7: Device according to one or more of the preceding embodiments, wherein the threaded element, in particular a distal end of the threaded element, is displaceable beyond the distal end of the shaft by a screwing motion.

Embodiment 8: Device according to one or more of the preceding embodiments, wherein the threaded element is a threaded sleeve.

Embodiment 9: Device according to the preceding embodiment, wherein the threaded element, in particular the threaded sleeve, features a division of the thread into two partial threads.

Embodiment 10: Device according to the preceding embodiment, wherein the two partial threads merge into one another, or wherein the two partial threads comprise an axial distance between each other.

Embodiment 11: Device according to one or both of the two preceding embodiments, wherein the two partial threads have opposite directions of rotation.

Embodiment 12: Device according to one or more of the preceding embodiments, wherein the threaded element is a union nut.

Embodiment 13: Device according to the preceding embodiment, wherein one side of the threaded element, in particular the union nut, comprises a reduced diameter of its central passage (shoulder).

Embodiment 14: Device according to the preceding embodiment, wherein the threaded element, in particular the union nut, is arranged such that its shoulder faces away from the distal end of the shaft.

Embodiment 15: Device according to one or more of the preceding embodiments, wherein the shaft comprises a widening (collar) in the vicinity of its distal end.

Embodiment 16: Device according to one or more of the preceding embodiments, wherein a threaded element, in particular a union nut, is arranged such that a pulling, directed to the distal end of the shaft, of the threaded element from the shaft is prevented by a positive fit between a collar of the shaft and a shoulder of the threaded element.

Embodiment 17: Device according to one or more of the preceding embodiments, wherein a threaded element, in particular a union nut, is configured such that, as seen in the longitudinal direction, it embraces a collar of the shaft from both sides, so that there is no significant or no axial displaceability of the threaded element.

Embodiment 18: Device according to one or more of the preceding embodiments, wherein the shaft is an actuating shaft.

Embodiment 19: Device according to the preceding embodiment, wherein the distal end of the shaft coincides with the distal end of the actuating shaft.

Embodiment 20: Device according to one or more of the preceding embodiments, wherein the shaft comprises an actuating shaft and an adapter piece connected thereto in the longitudinal direction.

Embodiment 21: Device according to the preceding embodiment, wherein the distal end of the shaft coincides with the distal end of the adapter piece.

Embodiment 22: Device according to one or more of the preceding embodiments, wherein the distal end of the shaft comprises, in its engagement section, teeth, which taper as seen in the longitudinal direction facing away from the shaft, so that, when engagement is established with a sleeve of an intramedullary nail which is arrangeable oppositely to the device in the longitudinal direction, with the sleeve comprising, in an engagement section, recesses, which taper in a corresponding manner as seen in the longitudinal direction facing away from the shaft, and, where applicable, merge into slots oriented in the longitudinal direction, the teeth can cause an expansion of the sleeve of the intramedullary nail, wherein an external thread is formed at the distal end of the shaft, and wherein the threaded element, in particular a threaded sleeve, comprises at a proximal end a first internal thread which engages with the external thread of the shaft, and comprises at a distal end a second internal thread which runs in the opposite direction to the first internal thread, and which is configured for connecting an intramedullary nail.

Embodiment 23: Device according to the preceding embodiment, wherein the teeth of the engagement section taper at a pointed angle as seen in the longitudinal direction facing away from the shaft.

Embodiment 24: Device according to one or both of the two preceding embodiments, wherein there exists a longitudinal axial distance between the first internal thread and the second internal thread.

Embodiment 25: Device according to one or more of the three preceding embodiments, wherein the shaft comprises a first marking for setting a first longitudinal axial position of the threaded element, in which a longitudinal axial and rotatably fixed engagement between the engagement section of the shaft and the engagement section of the intramedullary nail is present.

Embodiment 26: Device according to one or more of the four preceding embodiments, wherein the shaft comprises a second marking for setting a second longitudinal axial position of the threaded element, in which the engagement section of the shaft and the engagement section of the intramedullary nail are tensioned against each other in the longitudinal axial direction in such a way that the sleeve of the intramedullary nail is expanded.

Embodiment 27: Device according to one or more of the five preceding embodiments, wherein the threaded element comprises a section with a reduced diameter of its central passage (shoulder), which prevents the threaded element from being pulled off the shaft in the direction of the distal end of the shaft.

Embodiment 28: Device according to one or more of the six preceding embodiments, wherein the shaft comprises a widening (collar) in the vicinity of its distal end, which prevents the threaded element from being pulled off the shaft in the direction of the distal end of the shaft.

Embodiment 29: Device according to both of the two preceding embodiments, wherein the collar of the shaft is provided as a stop for the shoulder of the threaded element.

Embodiment 30: Threaded element which is configured to be arranged at the distal end of the shaft of a device according to embodiment 1, and which is rotatable relative to the shaft about the axial longitudinal direction, and which is configured to be screwed onto a complementary thread of an intramedullary nail which is arrangeable oppositely to the device in the longitudinal direction.

Embodiment 31: Set comprising a device according to embodiment 1 and a threaded element according to embodiment 30, for providing a device according to one or more of the preceding embodiments 2 to 29.

Embodiment 32: Device according to one or more of the preceding embodiments 1 to 29, or threaded element according to embodiment 30, wherein the device or threaded element further comprises at least one of the features discussed in the above description.

Embodiment 33: Device according to one or more of the preceding embodiments 1 to 29, or threaded element according to embodiment 30, wherein the device or threaded element comprises the additional features of at least one of the embodiments described in the above description.

Embodiment 34: Device according to one or more of the preceding embodiments 1 to 29, or threaded element according to embodiment 30, wherein the device or threaded element is configured according to one of the embodiments described in the above description.

Embodiment 35: Device according to one or more of the preceding embodiments 1 to 29, or threaded element according to embodiment 30, wherein the device or threaded element comprises the additional features of at least one further of the preceding embodiments.

1-15. (canceled)

16. A device for actuating an intramedullary nail, wherein the device extends in an axial longitudinal direction and comprises a proximal end and a distal end, the device further comprising:
a shaft assigned to an actuating element, wherein the distal end of the shaft coincides with the distal end of the device, and wherein the shaft comprises, at the distal end, an engagement section configured for engaging and rotating or retaining a complementary engagement section of the intramedullary nail, which is arrangeable oppositely to the device in the longitudinal direction; and
a threaded element arranged at the distal end of the shaft, which is rotatable relative to the shaft about the longitudinal direction, and which is screwable onto a complementary thread of the intramedullary nail.

17. The device according to claim 16, wherein the threaded element is a sleeve-like element which surrounds the shaft at the distal end.

18. The device according to claim 16, wherein the threaded element is displaceable relative to the shaft in the longitudinal direction.

19. The device according to claim 16,
wherein the distal end of the shaft comprises an external thread, and
wherein the threaded element comprises a corresponding internal thread, and is displaceable relative to the distal end of the shaft along the longitudinal direction by a screwing motion.

20. The device according to claim 19, wherein the threaded element is displaceable by the screwing motion beyond the distal end of the shaft.

21. The device according to claim 16, wherein the threaded element is a threaded sleeve or a union nut.

22. The device according to claim 16,
wherein the threaded element features a division of the thread into two partial threads, and
wherein the two partial threads are configured to merge into one another, or comprise an axial distance between each other.

23. The device according to claim 22, wherein the two partial threads have the same direction of rotation, or have opposite directions of rotation.

24. The device according to claim 16, wherein one side of the threaded element comprises a reduced diameter relative to a central passage.

25. The device according to claim 24, wherein the threaded element is arranged such that a shoulder faces away from the distal end of the shaft.

26. The device according to claim 24,
wherein the shaft comprises, in a vicinity of the distal end, a collar, and
wherein the threaded element is configured such that a pulling, directed to the distal end of the shaft, of the threaded element from the shaft is prevented by a positive fit between the collar of the shaft and a shoulder of the threaded element.

27. The device according to claim 16,
wherein the distal end of the shaft comprises, in the engagement section, teeth, which taper as seen in the longitudinal direction facing away from the shaft, so that, when an engagement is established with a sleeve of the intramedullary nail comprising, in the engagement section, recesses, which taper in a corresponding manner as seen in the longitudinal direction facing away from the shaft, and, where applicable, are configured to merge into slots oriented in the longitudinal direction, the teeth causing an expansion of the sleeve of the intramedullary nail,
wherein an external thread is arranged at the distal end of the shaft, and
wherein the threaded element is a threaded sleeve, which sleeve comprises, at a proximal end, a first internal thread, which is configured to engage with the external thread of the shaft, and which comprises, at a distal end, a second internal thread, which runs in the opposite direction, and which is configured for connecting the intramedullary nail.

28. The device according to claim 27, wherein the shaft comprises a first marking configured for setting a first longitudinal axial position of the threaded element, in which a longitudinal axial and rotatably fixed engagement between the engagement section of the shaft and the engagement section of the intramedullary nail is present.

29. The device according to claim 28, wherein the shaft comprises a second marking configured for setting a second longitudinal axial position of the threaded element, in which the engagement section of the shaft and the engagement section of the intramedullary nail are tensioned against each other in the longitudinal axial direction in such that the sleeve of the intramedullary nail is expanded.
